(19) United States
(12) Patent Application Publication
(10) Pub. No.: US 2026/0157682 A1
(43) Pub. Date: Jun. 11, 2026

(54) System for Monitoring and Analyzing Fluid Flow in Glymphatic System Using Near Infrared Spectroscopy

(71) Applicant: The Johns Hopkins University, Baltimore, MD (US)

(72) Inventors: Clara A. Scholl, Columbia, MD (US); William G. Coon, Laurel, MD (US); J. Kent Werner, Jr., Bethesda, MD (US)

(21) Appl. No.: 19/236,696

(22) Filed: Jun. 12, 2025

Related U.S. Application Data
(60) Provisional application No. 63/683,056, filed on Aug. 14, 2024.

(51) Int. Cl. A61B 5/00; A61B 5/0205; A61B 5/026; A61B 5/1455; A61B 5/369

(52) U.S. Cl. CPC A61B 5/4058; A61B 5/0205; A61B 5/0261; A61B 5/1455; A61B 5/369; A61B 5/4812; A61B 5/6803

(57) ABSTRACT

A system for monitoring and analyzing fluid flow dynamics in a glymphatic system of a head of a user includes an illustration system and a detector system configured to be supported by the head of the user. The illustration system includes a near infrared light source providing a first optical signal to probe a portion of the head. The first optical signal includes multiple wavelength components corresponding to extinction coefficients of different fluid compositions (e.g., cerebrospinal fluid (CSF) and blood) in the portion of the head. The detector system includes a near infrared detector to receive a second optical signal from the portion of the head in response to the first optical signal. The detector system is configured to analyze the second optical signal and extract dynamical parameters about the portion of the head, such as the variation of relative compositions of the CSF and the blood.

### CROSS-REFERENCE TO RELATED APPLICATIONS

This application claims priority to U.S. Provisional Patent Application No. 63/683,056, filed Aug. 14, 2024, which is incorporated herein by reference in its entirety.

### STATEMENT OF GOVERNMENTAL INTEREST

This invention was made with Government support under contract number HU00012020057 awarded by Uniform Services University of the Health Science. The Government has certain rights in the invention.

### BACKGROUND

### Field

Embodiments generally relate to methods and systems of monitoring and analyzing fluid dynamics in glymphatic system, using near infrared spectroscopy technologies.

### Background Art

Fluid flow dynamics in the brain's ventricles, interstitial spaces, and perivascular spaces, known as the “glymphatic system,” play a crucial role in brain waste clearance. Healthy function of this complex fluid transporter, most active during sleep, is critical for maintaining neurological health, and is considered important for recovery after acute and chronic injury (e.g. concussion). Conventional technologies of monitoring brain fluid dynamics may involve invasive contrast agents (e.g., fluorescent dyes injected into cerebrospinal fluid (CSF) and/or may not be portable or amenable to long-term repeated monitoring (e.g., magnetic resonance imaging (MRI) methods).

### SUMMARY

Embodiments of methods and systems for monitoring and analyzing brain fluid dynamics using near infrared spectroscopy technologies are described herein.

In some embodiments, a system can include an illumination system and detector system. The illumination system can be configured to be supported by a head of a user and to transmit a first optical signal through a portion of the head. The portion can include a cerebrospinal fluid (CSF) and blood vessels, in which blood flows. The illumination system can include a first radiation source and a second radiation source. The first radiation source can be configured to generate a first wavelength that is greater than a wavelength of an isosbestic point of the CSF and the blood. The second radiation source can be configured to generate a second wavelength that is less than the wavelength of the isosbestic point. The detector system can be configured to be supported by the head, to receive a second optical signal returned from the glymphatic system in response to the first optical signal, and to extract dynamical parameters of the CSF and the blood based on the second optical signal.

In some embodiments, a method can include providing a first optical signal to a portion of a head of a user. The portion of the head can include a CSF and blood vessels, in which blood flows. The first optical signal can include a first component and a second component. The first component can have a first wavelength greater than a wavelength of an isosbestic point of the CSF and the blood. The second component can have a second wavelength less than the wavelength of the isosbestic point. The method can further include receiving a second optical signal from the glymphatic system in response to the first optical signal and extracting dynamical parameters of the CSF and the blood based on the second optical signal.

In some embodiments, a device can include a near-infrared light source configured to provide a first optical signal to a portion of a head of a user. The portion of the head can include a CSF and blood vessels, in which blood flows. The near-infrared light source can include a first laser and a second laser. The first laser can have a first wavelength greater than a wavelength of an isosbestic point of the CSF and the blood. The second laser can have a second wavelength less than the wavelength of the isosbestic point. The device can further include a detector configured to receive a second optical signal returned from the glymphatic system in response to the first optical signal. The device can further include a monitoring unit coupled to the near-infrared light source and the detector. The monitoring unit can be configured to monitor dynamics of the CSF and the blood based on the second optical signal.

These as well as additional features, functions, and details of various embodiments are described below. Similarly, corresponding and additional embodiments are also described below.

### BRIEF DESCRIPTION OF THE DRAWINGS/FIGURES

Aspects of this disclosure are best understood from the following detailed description when read with the accompanying figures. It is noted that, in accordance with the common practice in the industry, various features are not drawn to scale. In fact, the dimensions of the various features may be arbitrarily increased or reduced for clarity of discussion.

FIGS. 1A and 1B illustrate a system for monitoring and analyzing the brain fluid dynamics of a user, in accordance with some embodiments.

FIGS. 2A and 2B illustrate modules with lasers and detectors for monitoring and analyzing the brain fluid dynamics, in accordance with some embodiments.

FIG. 3 illustrates a schematic diagram of transmitting optical signals into the glymphatic system by a laser and receiving response signals by detectors arranged on a head at different distance to the laser, in accordance with some embodiments.

FIG. 4 illustrates a diagram of spectra of extinction coefficients for oxyhemoglobin (HbO), deoxyhemoglobin (Hb), and water (H₂O), in accordance with some embodiments.

FIGS. 5A and 5B illustrate diagrams of separated signals about water and blood in the glymphatic system, in accordance with some embodiments.

FIG. 6 illustrates a schematic diagram of a system including four IR sources and eight IR detectors for monitoring and analyzing the brain fluid dynamics, in accordance with some embodiments.

FIG. 7 illustrates a system for synchronizing and processing data collected by electroencephalogram (EEG) and near infrared spectroscopy (NIRS) for monitoring and analyzing the brain fluid dynamics, in accordance with some embodiments.

FIG. 8 illustrates a flowchart of a method for monitoring and analyzing the brain fluid dynamics, in accordance with some embodiments.

FIG. 9 is an example computing system useful for implementing various aspects, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the process for performing a first operation and performing a second operation in the description that follows can include embodiments in which the first and second operations are performed in sequence, and can also include embodiments in which additional operations can be performed between the first and second operations, such that the second operation is not performed right after the first operation. In addition, the present disclosure can repeat reference numerals and/or letters in the various examples. This repetition does not in itself dictate a relationship between the various embodiments and/or configurations discussed.

Spatially relative terms, such as “beneath,” “below,” “lower,” “above,” “upper,” and the like can be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus can be otherwise oriented (rotated 90 degrees or at other orientations) and the spatially relative descriptors used herein can likewise be interpreted accordingly.

It is noted that references in the specification to “one embodiment,” “an embodiment,” “an example embodiment,” “exemplary,” etc., indicate that the embodiment described can include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to affect such feature, structure or characteristic in connection with other embodiments whether or not explicitly described.

It is to be understood that the phraseology or terminology herein is for the purpose of description and not of limitation, such that the terminology or phraseology of the present specification is to be interpreted by those skilled in relevant art(s) in light of the teachings herein.

In some embodiments, the terms “about” and “substantially” can indicate a value of a given quantity that varies within 5% of the value (e.g., ±1%, ±2%, ±3%, ±4%, ±5% of the value). These values are merely examples and are not intended to be limiting. The terms “about” and “substantially” can refer to a percentage of the values as interpreted by those skilled in relevant art(s) in light of the teachings herein.

The glymphatic system is a waste clearance system in the brain and includes the brain's ventricles, interstitial spaces, and perivascular spaces. The cerebrospinal fluid (CSF) flowing in these spaces is a water-dominated fluid and plays a crucial role in brain waste clearance. Probing the dynamics of the CSF can provide important information about the condition and status of the brain. However, in the complex environment of a human head, separating dynamical parameters of the CSF from those of other fluid can be challenging. For example, blood vessels are distributed in the brain's ventricles, interstitial spaces, and perivascular spaces and are surrounded by the CSF. Blood flowing in the blood vessels includes different compositions that vary dynamically. For example, hemoglobin, the protein in the blood that facilitates the transportation of oxygen in red blood cells, can include oxyhemoglobin (HbO) and deoxyhemoglobin (Hb). To distinguish the information about the CSF and the blood, conventional technologies of monitoring brain fluid dynamics require invasive contrast agents (e.g., fluorescent dyes injected into the CSF) and/or are not portable or amenable to long-term repeated monitoring (e.g., magnetic resonance imaging (MRI) methods)).

The embodiments described herein are directed to overcoming the challenges mentioned above. In particular, the embodiments can employ a near infrared (NIR) spectroscopic scheme to monitor and analyze brain fluid dynamics in a non-invasive manner, involving portable devices to facilitate long-term repeated monitoring in real time. In some embodiments, a system for monitoring and analyzing the fluid dynamics in a glymphatic system of a user can include an illumination system and a detector system configured to be supported by a head of the user. The illumination system can include a near infrared light source providing a first optical signal to probe a portion of the head of the user. The first optical signal can include multiple wavelength components corresponding to extinction coefficients of different compositions (e.g., CSF and blood) in the portion of the head. The detector system can include a near infrared detector to receive a second optical signal from the portion of the head in response to the first optical signal. The detector system is configured to analyze the second optical signal and extract dynamical parameters of the CSF and the blood, such as the variation of relative ratio of the CSF and the blood in the portion of the head.

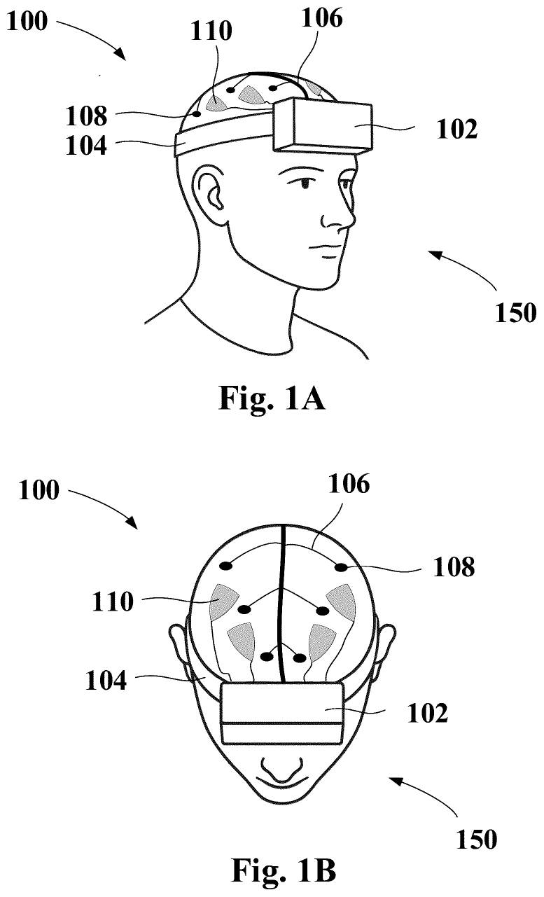

FIGS. 1A and 1B illustrate a system for monitoring and analyzing the brain fluid dynamics of a user, in accordance with some embodiments. For example, a system 100 can be used for monitoring and analyzing the brain fluid dynamics of a user 150. In particular, FIGS. 1A and 1B are, respectively, a front-side view and a front-top view about user 150 using system 100. System 100 can comprise a head-mounted device. The head-mounted device can comprise a control unit 102 and a belt 104 connected with control unit 102 for fixing control unit 102 on the head of user 150 (e.g., on the forehead of user 150). System 100 can also comprise infrared (IR) modules 110 attached to the head of user 150 and electrically coupled with control unit 102 via electrical cables 106. In some embodiments, system 100 can further comprise electroencephalography (EEG) electrodes 108 attached to the head of user 150 and electrically coupled with control unit 102 via electrical cables 106.

In some embodiments, control unit 102 can comprise an electronic circuit for controlling IR modules 110 and/or EEG electrodes 108. For example, control unit 102 can control IR modules 110 and/or EEG electrodes 108 to send probing signals to the head of user 150. Control unit 102 can also control IR modules 110 and/or EEG electrodes 108 to receive responding signals from the head of user 150. In some embodiments, control unit 102 can also include a memory circuit (e.g., a flash memory, a static random-access memory (SRAM), a dynamic random-access memory (DRAM), etc.) for storing data about the signals and controlling commands. In some embodiments, control unit 102 can also include a processing circuit (e.g., a micro-processor) to process the signals received by IR modules 110 and/or EEG electrodes 108. In some embodiments, control unit 102 can further include communication circuits to send and receive date from external devices, such as a computer. For example, control unit 102 can communicate with the external devices by wireless schemes (e.g., Bluetooth, Wi-Fi, optical, RF, etc.) or by electrical cables (e.g., universal serial bus (USB) cables). In some embodiments, system 100 can include the external devices, such as the computer to process and display the data about the signals.

In some embodiments, EEG electrodes 108 can include electrodes configured to measure electrical activities of the brain of user 150. For example, EEG electrodes 108 can collect signals indicating different sleeping stages of user 150. In some embodiments, EEG electrodes 108 can collect signals for diagnosing various neurological conditions of user 150, such as epilepsy, brain damage, sleep disorders, etc. In some embodiments, EEG electrodes 108 can be arranged in a symmetrical manner on the head of user 150. In some embodiments, EEG electrodes 108 can be arranged on locations of the head of user 150 that are in proximity to certain regions of the brain to be monitored and analyzed.

In some embodiments, system 100 can include a number of IR modules 110. For example, system 100 can include one, two, three, four, five, six IR modules 110. System 100 can also include more than six IR modules 110. In some embodiments, IR modules 110 can be arranged in a symmetrical manner on the head of user 150. In some embodiments, IR modules 110 can be arranged on locations of the head of user 150 that are in proximity to certain regions of the brain to be monitored and analyzed. Each of the IR modules 110 can include one or more IR sources and one or more IR detectors.

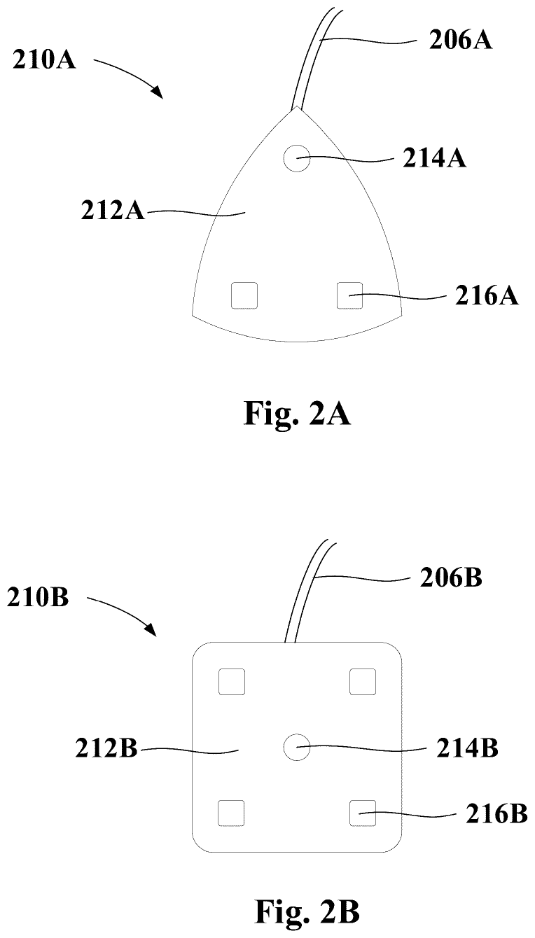

FIGS. 2A and 2B illustrate embodiments of IR modules 210A and 210B, respectively. Modules 210A or 210B can be one of IR modules 110 of system 100 as shown in FIGS. 1A and 1B. As shown in FIG. 2A, module 210A can include an IR source 214A and two IR detectors 216A on a substrate 212A. IR source 214A and two IR detectors 216A can be electrically connected to an electrical cable 206A, which can be one of electrical cables 106 as shown in FIGS. 1A and 1B. IR source 214A can receive control commands from control unit 102 by electrical cable 206A, and IR detectors 216A can send data to control unit 102 by electrical cable 206. In some embodiments, electrical cable 206A can also include power cables that supply electricity for IR source 214A and IR detectors 216A to function.

IR source 214A source can include optical elements providing probing optical signals into a portion of the head of user 150 to probe the glymphatic system of user 150. In particular, the probing optical signals include different wavelength components that are sensitive to different compositions of the fluid in the portion of the head of user 150. For example, the optical elements can include IR lasers with different wavelengths in the NIR range, and the probing optical signals can be IR laser beams with the different wavelengths. In some embodiments, the IR laser beams can have the same intensity. In some embodiments, the IR laser beams can have different intensities according to the controlling commands provided by control unit 102. In some embodiments, the IR laser beams can have varying intensities according to the controlling commands provided by control unit 102. In some embodiments, the IR lasers can be semiconductor IR lasers, such as diode lasers, edge-emitting lasers, surface emitting lasers, quantum cascade lasers, etc., or a combination thereof.

IR detectors 216A are configured to receive responding optical signals from the portion of the head of user 150, in response to the probing optical signals provided by IR source 214A. Each IR detector 216 can include a number of IR sensors corresponding to the different wavelength components of the probing optical signals. For example, if the probing optical signals include two wavelength components, IR detector 216 can include two IR sensors having different spectra of sensitivity corresponding to the two wavelength components. In some embodiments, the IR sensors can be semiconductor photodiodes, such as PN photodiodes, PIN photodiodes, Schottkey photodiodes, avalanche photodiodes, etc., or a combination thereof.

In some embodiments, substrate 212A can have a triangular shape. In some embodiments, IR source 214A can be arranged at a location close to a corner of substrate 212A. In some embodiments, IR detectors 216A can be arranged at a same distance from IR source 214A. In some embodiments, IR detectors 216A can be arranged at different distances from IR source 214A. In some embodiments, IR detectors 216A of an IR module can also receive responding optical signals from the glymphatic system of user 150, in response to the probing optical signals provided by IR sources 214A of other IR modules.

As shown in FIG. 2B, module 210B can include an IR source 214B and four IR detectors 216B on a substrate 212B and electrically coupled to an electrical cable 206B. The discussion of elements in FIG. 2A with similar annotations can apply to FIG. 2B, unless mentioned otherwise. In some embodiments, substrate 212B can have a rectangular shape. In some embodiments, IR source 214B can be arranged at a location around a center of substrate 212B. In some embodiments, IR detectors 216B can be arranged at a same distance from IR source 214B. In some embodiments, IR detectors 216B can be arranged at different distances from IR source 214B.

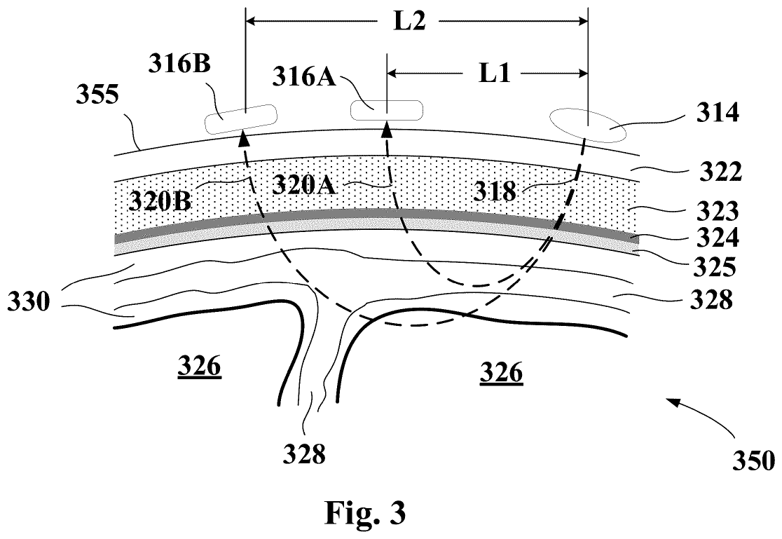

FIG. 3 illustrates a schematic diagram of transmitting probing optical signals by an IR source into the glymphatic system of a head of a user and receiving responding optical signals by IR detectors arranged on the head at different distances to the IR source, in accordance with some embodiments. For example, an IR source 314, a first IR detector 316A, and a second IR detector 316B can be arranged on a surface 355 of a head 350 of a user (e.g., user 150 as shown in FIGS. 1A and 1B). IR source 314 can be the same as IR source 214A or IR source 214B as shown in FIGS. 2A and 2B. Similarly, first and second IR detectors 316A/B can be the same as IR detectors 216A and 216B. As shown in FIG. 3, under surface 355, head 350 includes scalp 322, skull 323, dura mater 324, and arachnoid mater 325 that enclose the cranial cavity housing brain 326. The space between brain 326 and arachnoid mater 325 is subarachnoid space 330, which is a part of the glymphatic system and is filled with CSF that includes mostly water. There are blood vessels 328 distributed in and around brain 326. For example, blood vessels 328 can be located in subarachnoid space 330 or in sulci of brain 326. As discussed, the CSF plays a crucial role in brain waste clearance, and the ratio of the CSF and the blood in different portions of head 350 changes dynamically. IR source 314 and first and second IR detectors 316A/B can be used to collect information about the dynamics of the CSF and the blood in real time.

As shown in FIG. 3, IR source 314 provides a probing optical signal 318 through scalp 322, skull 323, dura mater 324, and arachnoid mater 325 into the cranial cavity. Probing optical signal 318 can diffuse along different paths through the glymphatic system, which generate different responding optical signals 320A and 320B returning back to surface 355, and received by first IR detector 316A and second IR detector 316B, respectively. Such curved paths of light diffusion in biological tissues are also referred to as a “photon banana.” First IR detector 316A and second IR detector 316B can be arranged at different distances with respect to IR source 314. For example, first IR detector 316A is placed at a first distance L1 and second IR detector 316B is placed at a second distance L2 greater than first distance L1. For example, first distance L1 can be about 30 mm, and second distance L2 can be about 40 mm. Such arrangement of first IR detector 316A and second IR detector 316B allows them to receive responding optical signals that carry information from different portion of the glymphatic system at different depths. For example, first IR detector 316A can receive responding optical signal 320A returned from shallower portion of the glymphatic and second IR detector 316B can receive responding optical signal 320B returned from deeper portion of the glymphatic system.

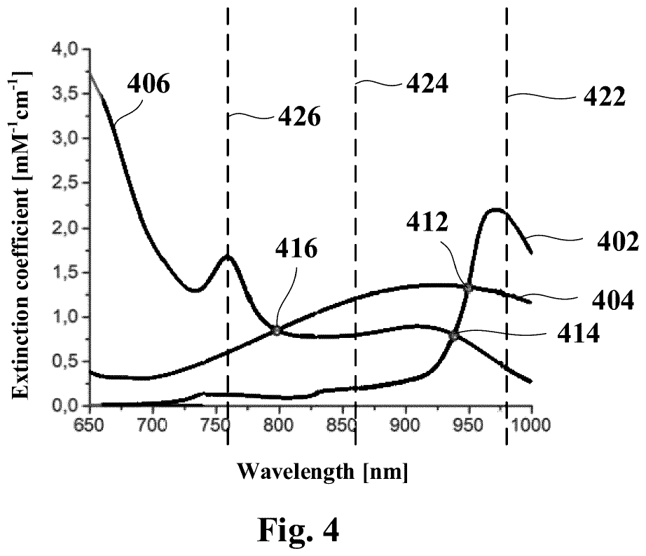

As discuss above, IR source 214A/B in FIGS. 2A and 2B can include IR lasers with different wavelengths within the NIR range to probe a portion of the head of the user and to examine the condition of CSF in the portion. FIG. 4 illustrates a diagram of spectra 402, 404, and 406 about extinction coefficients for water, HbO, and Hb, respectively. The crossing points of the three spectra are referred to as isosbestic points. In particular, an isosbestic point 412 of water and HbO is at a wavelength about 940 nm, an isosbestic point 414 of water and Hb is at a wavelength about 930 nm, and an isosbestic point 416 of Hb and HbO is at a wavelength about 805 nm. In order to resolve information about the CSF and the blood from the optical signals, the IR source can include the IR lasers having wavelengths spanning across the wavelengths of one or more isosbestic points.

In some embodiments, the IR source can include a first laser having a first wavelength greater than the wavelengths of isosbestic points 412 and 414. The IR source can also include a second laser having a second wavelength less than the wavelengths of isosbestic points 412 and 414. In some embodiments, the first and second wavelengths can have values indicated by dashed lines 422 and 424 in FIG. 4, respectively. For example, the first wavelength can be between about 960 nm and about 1000 nm, and the second wavelength can be between about 840 nm and about 910 nm. In particular, the first wavelength can be about 960 nm, about 970 nm, about 980 nm, about 990 nm, or about 1000 nm, and the second wavelength can be about 840 nm, about 850 nm, about 860 nm, about 870 nm, about 880 nm, about 890 nm, about 900 nm, or about 910 nm. Under such a combination of the first and second lasers, the IR source can provide a probing optical signal that includes a first wavelength component provided by the first laser and a second wavelength component provided by the second laser. Accordingly, water in CSF can have a high extinction coefficient in response to the first wavelength component and a low extinction coefficient in response to the second wavelength component. In contrast, blood, including Hb and HbO, can have a high extinction coefficient in response to the second wavelength component and a low extinction coefficient in response to the first wavelength component. Correspondingly, the IR detector receiving the response optical signal can have first and second IR sensors with different sensitivity spectra. In particular, the first IR sensor can be more sensitive around the first wavelength than around the second wavelength, and the second IR sensor can be more sensitive around the second wavelength than around the first wavelength. The information about water and blood can be extracted according to the modified Beer-Lambert transformation:

$$Δ {OD}_{λ} = {ε}_{λ}^{blood} \cdot Δ {c}^{blood} \cdot d \cdot D + {ε}_{λ}^{water} \cdot Δ {c}^{water} \cdot d \cdot D , \qquad ( 1 )$$

where ΔOD_{λ} is the total extinction coefficient of water and blood at wavelength

$$λ , {ε}_{λ}^{blood}$$

is the molar absorptivity of blood at wavelength λ, Δc^{blood} is the relative composition of blood, ε_{λ}^{water} is the molar absorptivity of water at wavelength λ, Δc^{water} is the relative composition of water, d is the depth of the portion of the head responding to the probe optical signal, and D is a differential pathlength factor that takes into account the shape of the photon banana between the IR source and the IR detector.

In some embodiments, the IR source can include two lasers having a combination of wavelengths different from the above. In particular, the first wavelength can be greater than the wavelength of isosbestic point 416, and the second wavelength can be less than the wavelength of isosbestic point 416. In some embodiments, the first and second wavelengths can have values indicated by dashed lines 424 and 426 in FIG. 4, respectively. For example, the first wavelength can be between about 820 nm and about 900 nm, and the second wavelength can be between about 700 nm and about 780 nm. In particular, the first wavelength can be about 820 nm, about 830 nm, about 840 nm, about 850 nm, about 860, about 870, about 880, about 890, or about 900 nm, and the second wavelength can be about 700 nm, about 710 nm, about 720 nm, about 730 nm, about 740 nm, about 750 nm, about 760 nm, about 770, or about 780. Accordingly, the IR detector can include two IR sensors with the first IR having a sensitivity corresponding to the first wavelength and the second IR having a sensitivity corresponding to the second wavelength. Under the first and second wavelengths, the extinction coefficient of water is relatively low compared with that of blood. More importantly, under the first wavelength, the extinction coefficient of HbO is greater than that of Hb, and under the second wavelength, the extinction coefficient of HbO is less than that of Hb. Therefore, the above wavelength combination allows the separation of Hb and HbO according to the modified Beer-Lambert transformation:

$$Δ {OD}_{λ} = {ε}_{λ}^{Hb} \cdot Δ {c}^{Hb} \cdot d \cdot D + {ε}_{λ}^{HbO} \cdot Δ {c}^{HbO} \cdot d \cdot D , \qquad ( 2 )$$

where ΔOD_{λ} is the total extinction coefficient of blood at wavelength λ,

$${ε}_{λ}^{Hb}$$

is the molar absorptivity of Hb at wavelength λ, Δc^{Hb} is the relative composition of Hb, ε_{λ}^{HbO} is the molar absorptivity of HbO, Δc^{HbO} is the relative composition of HbO, d is the depth of the portion of the head responding to the probe optical signal, and D is a differential pathlength factor that takes into account the shape of the photon banana between the IR source and the IR detector.

In some embodiments, the IR source can include three lasers with three different wavelengths, which can have values indicated by the dashed lines 422, 424, and 426 in FIG. 4, respectively. For example, the IR source can include a first laser having a first wavelength between about 960 nm and about 1000 nm, a second laser having a second wavelength between about 820 nm and about 900 nm, and a third laser having a third wavelength between about 700 nm and about 780 nm. Accordingly, the IR detector can include three IR sensors with three different sensitivities corresponding to the first, second, and third wavelengths. Such a combination allows the separation of water, HbO, and Hb according to the modified Beer-Lambert transformation:

$$Δ {OD}_{λ} = {ε}_{λ}^{water} \cdot Δ {c}^{water} \cdot d \cdot D + {ε}_{λ}^{Hb} \cdot Δ {c}^{Hb} \cdot d \cdot D + {ε}_{λ}^{HbO} \cdot Δ {c}^{HbO} \cdot d \cdot D . \qquad ( 3 )$$

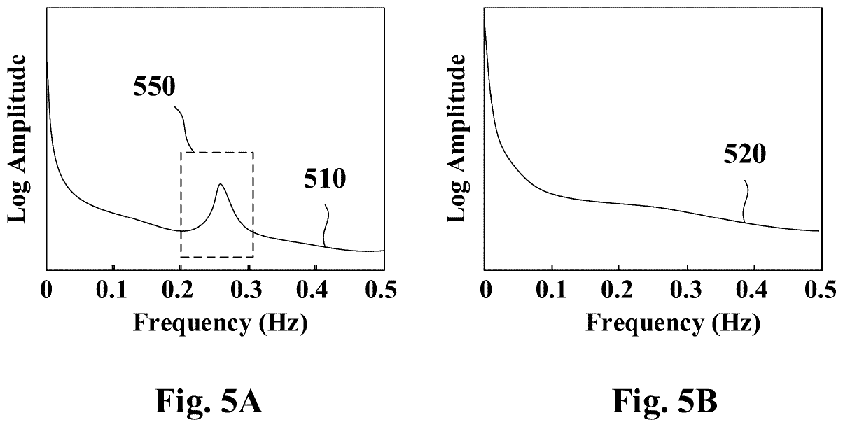

In some embodiments, the information about the water-dominated CSF and the blood can be separately shown in the frequency domain after applying the modified Beer-Lambert transformation on the responding optical signals. FIGS. 5A and 5B respectively illustrate the information about the blood and the CSF, according to some embodiments. In particular, a spectrum 510 as shown in FIG. 5A include a peak as a signature of HbO in a range 550 between about 0.2 Hz and about 0.3 Hz. In comparison, a spectrum 520 as shown in FIG. 5B about water do not have such a peak around similar frequency range. The relative amplitudes between spectra 510 and 520 in FIGS. 5A and 5B provide the information about the relative compositions of HbO and the CSF at a probed portion of a head of a user, measured by the IR source and the IR detector.

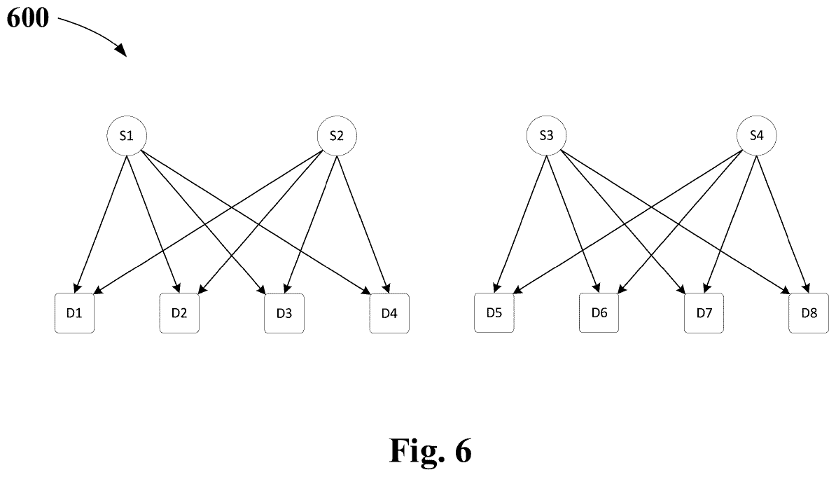

Referring to FIGS. 1A and 1B, where system 100 can include four IR modules 110, each can include an IR source and two IR detectors (such as IR source 214A and IR detectors 216A, as shown in FIG. 2A). The four IR sources can form an illumination system. Each of the IR sources can include two IR lasers with two different wavelengths. The eight IR detectors together with control unit 110 can form a detector system. Each of the IR detectors can include two IR sensors having sensitivities corresponding to the two different wavelengths. The four IR modules 110 are arranged in a symmetrical manner, such that two of them in a first group are on a left hand side of the head of user 150, and two others in a second group are on a right hand side of the head of user 150. The two IR modules 110 on the left hand side include two IR sources S1 and S2 and four IR detectors D1, D2, D3, and D4, as illustrated in FIG. 6. Similarly, the two IR modules 110 on the right hand side include two IR sources S3 and S4 and four IR detectors D5, D6, D7, and D8. The IR sources and IR detectors in a same group are placed sufficiently close such that the IR detectors can receive responding optical signals from the glymphatic system in response to the probing optical signals sent by the IR sources. For example, IR detectors D1, D2, D3, and D4 can receive responding optical signals from the glymphatic system in response to the probing optical signals provided by the IR sources S1 and S2, and IR detectors D5, D6, D7, and D8 can receive responding optical signals from the glymphatic system in response to the probing optical signals provided by the IR sources S3 and S4. The IR sources and IR detectors of different groups are placed sufficiently apart such that IR detectors of one group cannot receive responding optical signals from the glymphatic system in response to the probing optical signals provided by the IR sources of another group. Therefore, there are 16 signal paths of probing and response in system 600 (e.g., S1 to D1, S3 to D8, S4 to D6, etc.). Given that each signal path includes two different wavelength channels, in system 600, there are 32 channels in total. Among the 16 signal paths, there can be three different source-detector distances. For example, S1 to D1 and S1 to D2 can have a first source-detector distance, S1 to D3 can have a second source-detector distance greater than the first source-detector distance, and S1 to D4 can have a third source-detector distance greater than the second source-detector distance. In some embodiments, the first source-detector distance can be about 2.5 cm, the second source-detector distance can be about 4 cm, and the third source-detector distance can be about 5 cm.

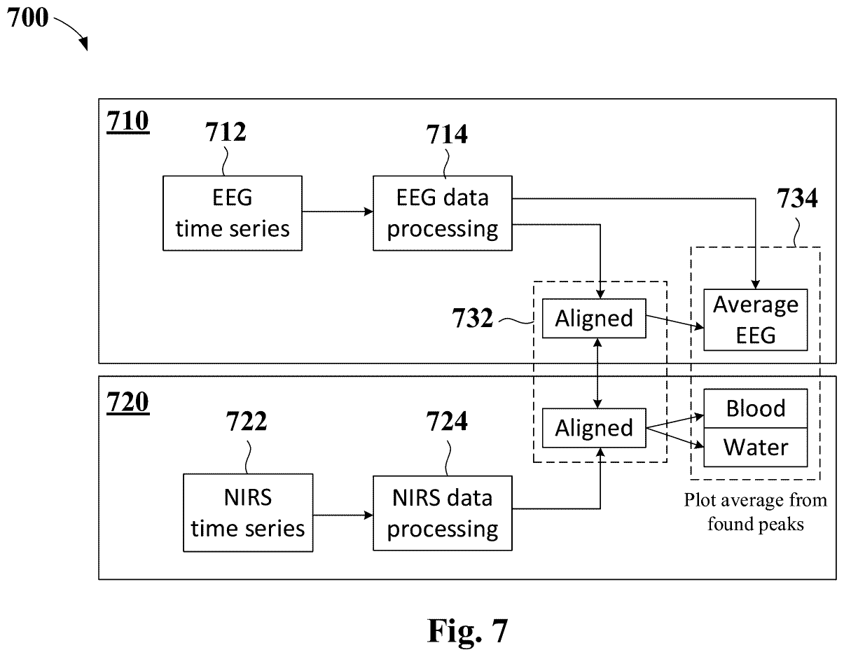

In some embodiments, using system 100 as shown in FIGS. 1A and 1B, data collected by IR modules 110 can be synchronized with data collected by EEG electrodes to analyze the dynamics of the glymphatic system. FIG. 7 illustrates a system 700 for monitoring and analyzing the glymphatic system using both near infrared spectroscopy (NIRS) and EEG. System 700 can include an EEG processing module 710 and an NIRS processing module 720. EEG processing module 710 can include an EEG time series unit 712 that receives EEG data collected by EEG electrodes (e.g., EEG electrodes 108 as shown in FIGS. 1A and 1B). The EEG electrodes and EEG processing module 710 can be parts of an EEG monitoring system. EEG processing module 710 can further include an EGG data processing unit 714 performing functions such as band pass filtering, data projecting, data enveloping, data normalization, etc. NIRS processing module 720 can include an NIRS time series unit 722 that receives NIRS data collected by IR modules (e.g., IR modules 110 as shown in FIGS. 1A and 1B). NIRS processing module 710 can further include an NIRS data processing unit 724 performing functions such as artifact rejection, time series separation, time-dependent derivation, band pass filtering, data normalization, etc. After processing the EEG data and the NIRS data, an EEG and NIRS data alignment unit 732 shared by EEG processing module 710 and NIRS processing module 710 can align the EEG data and the NIRS data to identify their signatures (e.g., peaks). An EEG and NIRS data display unit 734, also shared by EEG processing module 710 and NIRS processing module 710 can further display the real-time variation of the blood and the CSF with respect to the real-time information provided by the averaged EEG data. For example, the averaged EEG data can provide information about the user's stages of sleep (e.g., wake, N1 (a stage of light sleep), N2 (a stage of deeper sleep), N3 (a stage of deepest sleep), and rapid eye movement (REM)), and the variation of the blood and the CSF at different stages of sleep can be displayed by system 700.

In some embodiments, system 700 can be a part of system 100 as shown in FIGS. 1A and 1B. For example, system 700 can be incorporated in control unit 102. In some embodiments, system 700 and system 100 can be separated and can communicate with each other. For example, system 700 can be a computer with its own processing unit, memory, hard drive, and display, and can communicate with control unit 102 by electrical cables or wireless schemes.

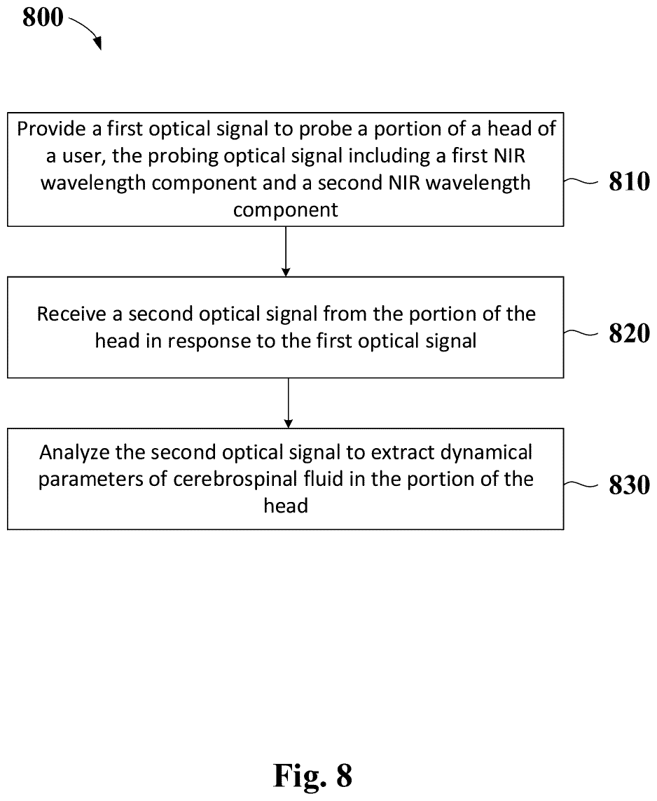

FIG. 8 illustrates a method 800 for monitoring and analyzing the brain fluid dynamics of a user, in accordance with some embodiments. Method 800 can begin with operation 810 to provide a first optical signal to probe a portion of a head of the user. In some embodiments, the first optical signal can include near infrared (NIR) laser beams with multiple wavelength components. In some embodiments, the first optical signal can be provided by an NIR source (e.g., IR sources 214A and 214B as shown in FIGS. 2A and 2B) with multiple NIR lasers. For example, the first optical signal can include a first component with a first wavelength and a second component with a second wavelength. The first and second wavelengths are chosen corresponding to extinction coefficient spectra of different compositions in the portion of the head. In particular, the first wavelength can be greater than a wavelength of an isosbestic point of the CSF and the blood in the portion of the head, and the second wavelength can be less than the wavelength of the isosbestic point. In some embodiments, the first and second components can be continuous-wave signals.

Referring to FIG. 8, method 800 can continue with operation 820 to receive a second optical signal from a glymphatic system in response to the first optical signal. In some embodiments, the second optical signal can be received by an NIR detector (e.g., IR detectors 216A and 216B as shown in FIGS. 2A and 2B) with multiple NIR sensors having different sensitivities. Each of the NIR sensors can have a sensitivity corresponding to one of the wavelength components in the first optical signal.

Referring to FIG. 8, method 800 can continue with operation 830 of analyzing the second optical signal to extract dynamical parameters of the CSF and the blood in the portion of the head. In some embodiments, analyzing the second optical signal can include resolving information about the different compositions in the portion of the head. For example, analyzing the second optical signal can include extracting the relative compositions the CSF and the blood in the portion of the head, and/or extracting the relative compositions of Hb and HbO in the blood. In some embodiments, analyzing the second optical signal can include providing the dynamical parameters of the CSF and the blood in real-time. In some embodiments, analyzing the second optical signal can include synchronizing the second optical signal with EEG signals to monitor the dynamical parameters of the CSF and the blood at different sleeping stages of the user.

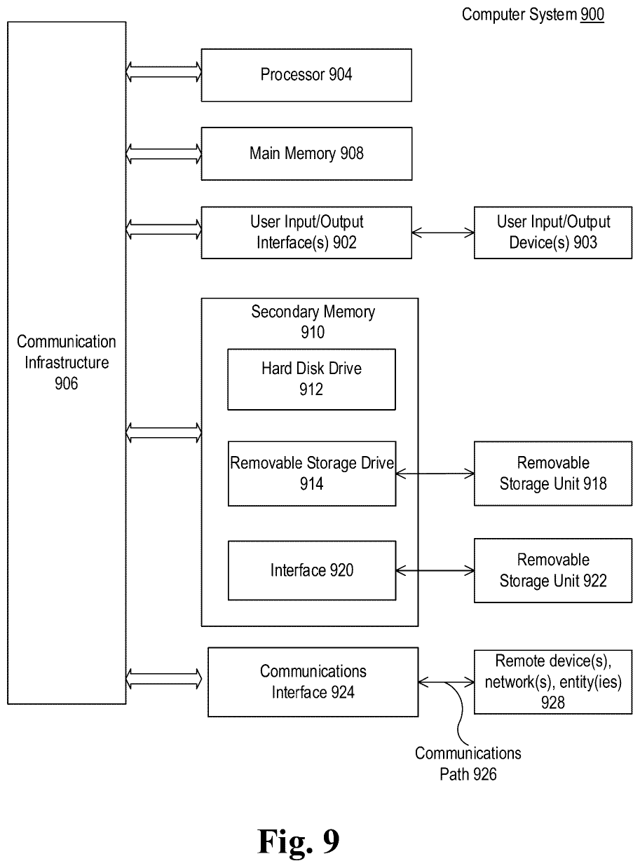

FIG. 9 is an example computer system 900 useful for implementing systems 100, 600, and/or 700, in accordance with aspects of the disclosure. Computer system 900 may be any computer capable of performing the functions described herein. For example, control unit 102 as shown in FIGS. 1A and 1B may be implemented using components of the computing system 900. EGG data processing unit 714, NIRS data processing unit 724, EEG and NIRS data alignment unit 732, and EEG and NIRS data display unit 734 as shown in FIG. 7 may also be implemented using components of the computing system 900.

Computer system 900 includes one or more processors (also called central processing units, or CPUs), such as a processor 904. Processor 904 is connected to a communication infrastructure or bus 906.

One or more processors 904 may each be a graphics processing unit (GPU). In an aspect, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 900 also includes user input/output device(s) 903, such as monitors, keyboards, pointing devices, etc., that communicate with communication infrastructure 906 through user input/output interface(s) 902.

Computer system 900 also includes a main or primary memory 908, such as random access memory (RAM). Main memory 908 may include one or more levels of cache. Main memory 908 has stored therein control logic (i.e., computer software) and/or data.

Computer system 900 may also include one or more secondary storage devices or memory 910. Secondary memory 910 may include, for example, a hard disk drive 912 and/or a removable storage device or drive 914. Removable storage drive 914 may be a floppy disk drive, a magnetic tape drive, a compact disk drive, an optical storage device, tape backup device, and/or any other storage device/drive.

Removable storage drive 914 may interact with a removable storage unit 918. Removable storage unit 918 includes a computer usable or readable storage device having stored thereon computer software (control logic) and/or data. Removable storage unit 918 may be a floppy disk, magnetic tape, compact disk, DVD, optical storage disk, and/any other computer data storage device. Removable storage drive 914 reads from and/or writes to removable storage unit 918 in a well-known manner.

According to an exemplary aspect, secondary memory 910 may include other means, instrumentalities or other approaches for allowing computer programs and/or other instructions and/or data to be accessed by computer system 900. Such means, instrumentalities or other approaches may include, for example, a removable storage unit 922 and an interface 920. Examples of the removable storage unit 922 and the interface 920 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 900 may further include a communication or network interface 924. Communication interface 924 enables computer system 900 to communicate and interact with any combination of remote devices, remote networks, remote entities, etc. (individually and collectively referenced by reference number 928). For example, communication interface 924 may allow Computer system 900 to communicate with remote devices 928 over communications path 926, which may be wired and/or wireless, and which may include any combination of LANs, WANs, the Internet, etc. Control logic and/or data may be transmitted to and from computer system 900 via communication path 926.

In some embodiments, a tangible, non-transitory apparatus or article of manufacture comprising a tangible, non-transitory computer useable or readable medium having control logic (software) stored thereon is also referred to herein as a computer program product or program storage device. This includes, but is not limited to, Computer system 900, main memory 908, secondary memory 910, and removable storage units 918 and 922, as well as tangible articles of manufacture embodying any combination of the foregoing. Such control logic, when executed by one or more data processing devices (such as computer system 900), causes such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 9. In particular, aspects can operate with software, hardware, and/or operating system implementations other than those described herein.

It is to be appreciated that the Detailed Description section, and not the Summary and Abstract sections (if any), is intended to be used to interpret the claims. The Summary and Abstract sections (if any) may set forth one or more but not all exemplary embodiments of the invention as contemplated by the inventor(s), and thus, are not intended to limit the invention or the appended claims in any way.

While the embodiments have been described herein with reference to exemplary embodiments for exemplary fields and applications, it should be understood that the embodiments are not limited thereto. Other embodiments and modifications thereto are possible, and are within the scope and spirit of the disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments may perform functional blocks, steps, operations, methods, etc. using orderings different than those described herein.

References herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

The breadth and scope of the embodiments should not be limited by any of the above-described exemplary embodiments, but should be defined only in accordance with the following claims and their equivalents.

What is claimed is:

1. A system comprising:
an illumination system configured to be supported by a head of a user and to transmit a first optical signal through a portion of the head, wherein the portion comprises a cerebrospinal fluid (CSF) and blood vessels in which blood flows, and wherein the illumination system comprises:
a first radiation source configured to generate a first wavelength that is greater than a wavelength of an isosbestic point of the CSF and the blood; and
a second radiation source configure to generate a second wavelength that is less than the wavelength of the isosbestic point; and
a detector system configured to be supported by the head, to receive a second optical signal returned from a glymphatic system of the user in response to the first optical signal, and to extract dynamical parameters of the CSF and the blood based on the second optical signal.

2. The system of claim 1, wherein:
the first wavelength is between about 960 nm and about 1000 nm; and
the second wavelength is between about 840 nm and about 910 nm.

3. The system of claim 1, wherein the detector system comprises a detector, wherein the detector comprises:
a first optical sensor, wherein a first response spectrum of the first optical sensor comprises a first sensitivity peak at the first wavelength; and
a second optical sensor, wherein a second response spectrum of the second optical sensor comprises a second sensitivity peak at the second wavelength.

4. The system of claim 1, wherein the dynamical parameters comprises relative compositions of the CSF and the blood.

5. The system of claim 1, wherein:
the blood comprises a first composition and a second composition;
the illumination system further comprises a third radiation source configured to generate a third wavelength;
the third wavelength is less than another wavelength of another isosbestic point of the first and second compositions; and
the second wavelength is greater than the another wavelength of the another isosbestic point.

6. The system of claim 5, wherein the detector system is further configured to extract dynamical parameters of the first and second compositions based on the second optical signal.

7. The system of claim 1, further comprising another illumination system configured to transmit a third optical signal through another portion of the head, wherein the detector system is further configured to receive a fourth optical signal returned from the another portion in response to the third optical signal.

8. The system of claim 1, wherein the detector system further comprises:
a first detector at a first distance from the illumination system; and
a second detector at a second distance from the illumination system, wherein the first and second distances are different.

9. The system of claim 8, wherein:
the first detector is further configured to receive the second optical signal returned from a first depth in the portion of the head;
the second detector is further configured to receive a third optical signal returned from a second depth in the portion of the head; and
the first depth is different from the second depth.

10. The system of claim 1, further comprising an electroencephalogram (EEG) monitoring system configured to determine different sleeping phases of the user, wherein the detector system is further configured to synchronize with the EEG monitoring system and to monitor temporal dynamics of the dynamical parameters of the CSF and the blood at the different sleeping phases.

11. The system of claim 1, wherein the detector system is further configured to perform a modified Beer-Lambert transformation to the second optical signal to isolate the dynamical parameters of the CSF and the blood.

12. A method comprising:
providing a first optical signal to a portion of a head of a user, wherein the portion comprises a cerebrospinal fluid (CSF) and blood vessels in which blood flows, and wherein the first optical signal comprises:
a first component having a first wavelength greater than a wavelength of an isosbestic point of the CSF and the blood; and
a second component having a second wavelength less than the wavelength of the isosbestic point;
receiving a second optical signal from the portion of the head in response to the first optical signal; and
extracting dynamical parameters of the CSF and the blood based on the second optical signal.

13. The method of claim 12, further comprising performing a modified Beer-Lambert transformation to the second optical signal to isolate the dynamical parameters of the CSF and the blood.

14. The method of claim 12, wherein the first and second components are continuous-wave signals.

15. The method of claim 12, further comprising receiving a third optical signal from the portion of the head in response to the first optical signal, wherein the second and third optical signals are returned from different depths in the portion of the head.

16. A device comprising:
a near-infrared light source configured to provide a first optical signal to a portion of a head of a user, wherein the portion comprises a cerebrospinal fluid (CSF) and blood vessels in which blood flows, and wherein the near-infrared light source comprises:
a first laser having a first wavelength greater than a wavelength of an isosbestic point of the CSF and the blood; and
a second laser having a second wavelength less than the wavelength of the isosbestic point;
a detector configured to receive a second optical signal returned from the portion of the head in response to the first optical signal; and
a monitoring unit coupled to the near-infrared light source and the detector, wherein the monitoring unit is configured to monitor dynamics of the CSF and the blood based on the second optical signal.

17. The device of claim 16, wherein the monitoring unit is further configured to monitor the CSF and the blood in real time.

18. The device of claim 16, wherein the wavelength of the isosbestic point is between about 800 nm and about 820 nm.

19. The device of claim 16, wherein the near-infrared light source and the detector are further configured to be supported by a head of a user of the device.

20. The device of claim 16, wherein the monitoring unit is further configured to isolate dynamical parameters of the CSF and the blood based on the second optical signal.
